(19) United States
(12) Patent Application Publication
(10) Pub. No.: US 2005/0038501 A1
(43) Pub. Date: Feb. 17, 2005

(54) Dynamic stent

(76) Inventors: James E. Moore JR., College Station, TX (US); Michael R. Moreno, College Station, TX (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/835,826

(22) Filed: Apr. 30, 2004

Related U.S. Application Data
(60) Provisional application No. 60/494,476, filed on Aug. 12, 2003.

(51) Int. Cl. A61F 2/06

(52) U.S. Cl. 623/1.19; 623/1.2; 623/1.15

(57) ABSTRACT

A stent (100) including a support frame (106) for supporting a vessel in a non-collapsed state is disclosed. The support frame (106) includes a degradable component (102) for at least initially supporting the vessel in the non-collapsed state when the stent (100) is first implanted in the vessel. The degradable component (102) is degradable after implantation such that support provided by the degradable component (102) decreases a selected amount after a predetermined time after implantation. The support frame (106) may further include a durable component (104) which is resistant to degradation such that support provided by the durable component (104) remains substantially constant after implantation.

### CROSS-REFERENCES TO RELATED APPLICATION

This application claims the benefit of U.S. Provisional Patent Application No. 60/494,476 filed on Aug. 12, 2003, the disclosure of which is hereby expressly incorporated by reference, and priority from the filing date of which is hereby claimed under 35 U.S.C. § 119(e).

### FIELD OF THE INVENTION

The present invention relates generally to stents, and more particularly to stents providing dynamic support of a vessel after implantation.

### BACKGROUND OF THE INVENTION

Stenting is a non-surgical treatment used with balloon angioplasty to treat coronary artery disease. Right after angioplasty has widened a coronary artery, a stent (one example being a small, expandable wire mesh tube) is inserted within the artery. The purpose of the stent is to help hold the newly treated artery open, reducing the risk of the artery re-closing (restenosis) over time.

Although stents have been widely used as solid mechanical, structural supports to maintain a vessel in a non-collapsed state following balloon angioplasty, they are not without their problems. Studies on the response of the artery wall to a stent demonstrate that the artery wall responds in distinct phases, displaying varying behaviors during certain time intervals after implantation. The earliest response, thrombus formation, is followed by ramping up inflammatory responses, smooth muscle cell proliferation, and finally, remodeling. Re-endothelization of the intima occurs on the time frame of weeks.

Research has demonstrated that stent design influences these actions through biomechanically mediated responses. For example, blood flow patterns dictate that platelet deposition is lowest when stent strut spacing is small, whereas endothelial cell regrowth is fastest when stent strut spacing is large. Stent-induced artery wall stresses (which depend heavily on strut configuration) also play a role in the inflammatory and proliferative responses. While each of these responses have distinctly different characteristic times of action, previously developed stents are either static and do not change over time, or are fully degradable and may fail to provide sufficient structural support for supporting the artery. Thus, there exists a need for a stent which is reliable, easy to manufacture, and which is dynamic such that the properties of the stent change over time to correspond to the changing responses and needs of the vessel.

### SUMMARY OF THE INVENTION

One embodiment of a stent formed in accordance with the present invention is disclosed. The stent includes a support frame for supporting a vessel in a non-collapsed state. The support frame includes a degradable component for at least initially supporting the vessel in the non-collapsed state when the stent is first implanted in the vessel. The degradable component is degradable after implantation such that support provided by the degradable component decreases a selected amount after a predetermined time after implantation. The support frame further includes a durable component for supporting the vessel in the non-collapsed state. The durable component is resistant to degradation over time such that support provided by the durable component remains substantially constant after implantation.

Another embodiment of a stent formed in accordance with the present invention for supporting a vessel in a non-collapsed state is disclosed. The stent includes a plurality of durable struts for supporting the vessel in the non-collapsed state. The stent further includes a plurality of temporary struts for initially aiding in the support of the vessel in the non-collapsed state. The temporary struts break down over time after implantation such that they no longer substantially aid in supporting the vessel in the non-collapsed state.

Still another embodiment of a stent formed in accordance with the present invention for supporting a vessel in a non-collapsed state is disclosed. The stent includes a support frame having a durable component and a degradable component, the support frame providing a variable level of support for supporting the vessel in the non-collapsed state. Upon implantation, the support frame initially provides a predetermined amount of support for supporting the vessel in the non-collapsed state. After implantation, the support frame changes due to exposure to environmental conditions such that after passage of a selected duration, the support frame provides a selected lessened amount of support for supporting the vessel in the non-collapsed state.

### BRIEF DESCRIPTION OF THE DRAWINGS

The foregoing aspects and many of the attendant advantages of this invention will become better understood by reference to the following detailed description, when taken in conjunction with the accompanying drawings, wherein:

FIG. 1 is a perspective view of one embodiment of a dynamic stent formed in accordance with the present invention, the dynamic stent including a permanent component and a temporary component;

FIG. 2 is a side view of the dynamic stent of FIG. 1;

FIG. 3 is a perspective view of the permanent component of the dynamic stent of FIG. 1;

FIG. 4 is a side view of the permanent component of the dynamic stent of FIG. 1;

FIG. 5 is a perspective view of the temporary component of the dynamic stent of FIG. 1;

FIG. 6 is a side view of the temporary component of the dynamic stent of FIG. 1; and

FIG. 7 is a side view of an alternate embodiment of a dynamic stent formed in accordance with the present invention, wherein the dynamic stent is formed so as to have a variable stiffness along the length of the dynamic stent.

### DETAILED DESCRIPTION OF THE PREFERRED EMBODIMENT

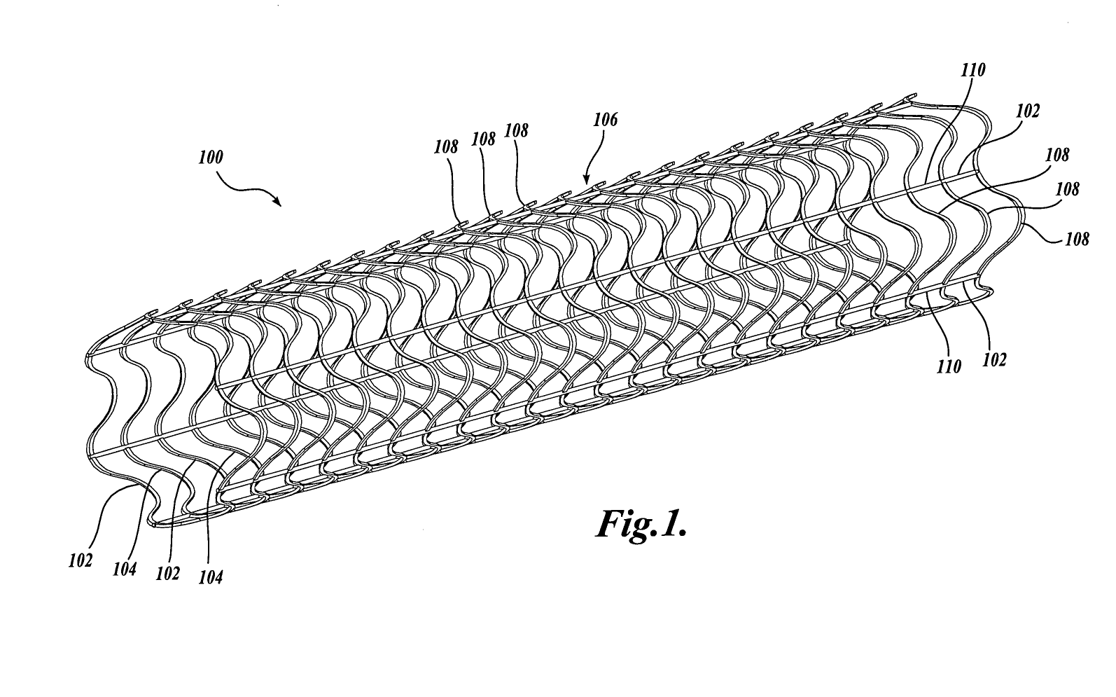

Referring to FIGS. 1-6, one embodiment of a dynamic stent 100 formed in accordance with the present invention is depicted. The dynamic stent 100 incorporates a hybrid structure having both a durable or permanent component 102 and a temporary component 104. The permanent component 102 is suitably a durable structure that remains in the artery for an extended period, such as for the life of the user. The temporary component 104 changes over time to accommodate the changing requirements of artery wall rehabilitation after stent implantation.

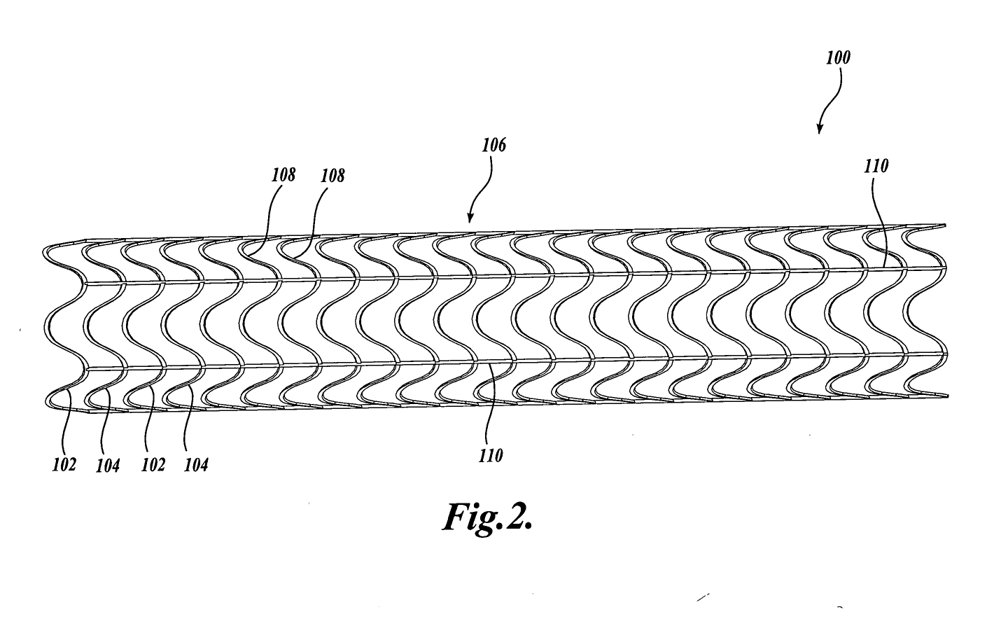

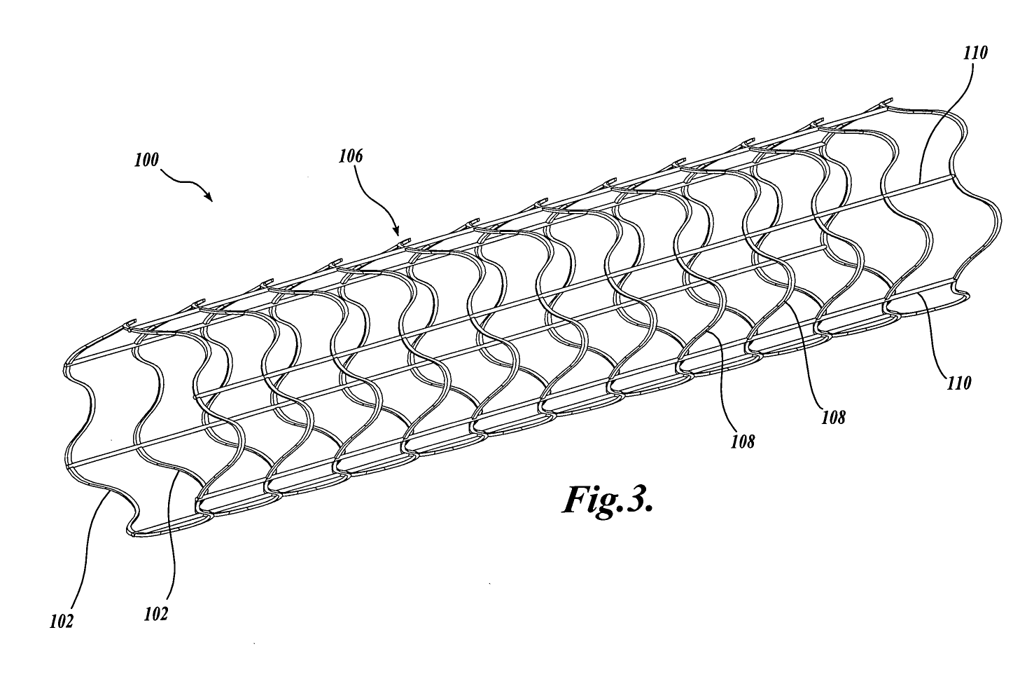

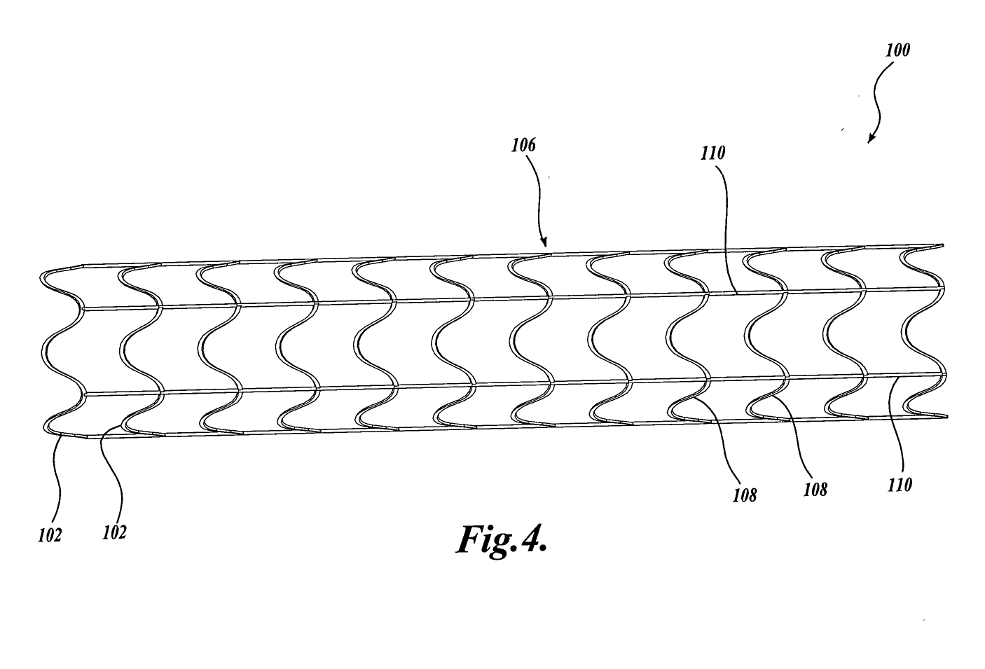

Moreover, in the early stages after implantation, both the permanent and temporary components 102 and 104 are present in the dynamic stent 100, such as shown in FIGS. 1 and 2, providing improved artery wall support through small strut spacing. This configuration serves to minimize platelet deposition and to hold back intimal flaps. As time passes, the temporary component 104 degrades away, eventually leaving just the permanent component 102, as shown in FIGS. 3 and 4. In this new phase of artery rehabilitation, the dynamic stent 100 has a rather sparse strut spacing which increases the shear stress on the artery wall, since shear stress depends heavily on strut spacing. This larger strut spacing is allowable because there is presumably less of a need to hold back intimal flaps once partial healing of the artery wall has occurred.

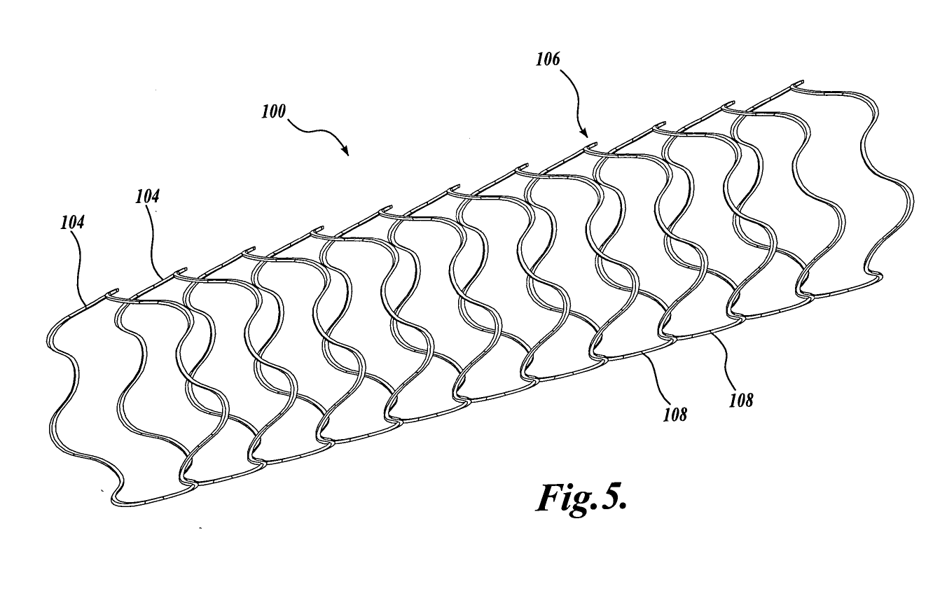

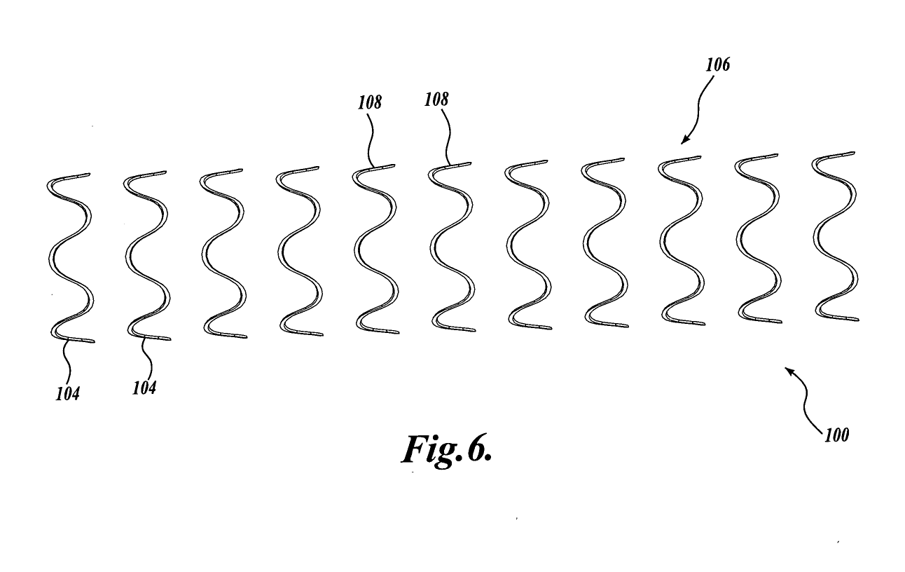

Referring to FIGS. 1 and 2, this detailed description will now focus upon the structure of the dynamic stent 100. As stated above, the dynamic stent 100 includes a permanent component 102 (best shown in FIGS. 3 and 4) and a temporary component 104 (best shown in FIGS. 5 and 6). The permanent and temporary components 102 and 104 are interwoven with one another, and in combination, form a support frame 106 for supporting an artery wall (not shown). The support frame 106 is tubular in shape providing a central lumen along its central longitudinal axis.

The support frame 106 includes a plurality of struts or annular links 108. The annular links 108 of the illustrated embodiment are sinusoidal in shape and are generally equally spaced along the length of the dynamic stent 100. The sinusoidal shape of the annular links 108 provides a blunt end profile for the dynamic stent 100 in order minimize the risk of puncturing the vessel and provides increased support of the artery wall over a straight annular link. Further, the sinusoidal shape of the annular links 108 permits the dynamic stent 100 to be expanded from a small diameter to a larger diameter once the dynamic stent is properly positioned within the artery. The dynamic stent 100 may be expanded by any suitable technique, such as by balloon expansion or self expansion.

Although a sinusoidal shape of the annular links 108 is described and depicted, it should be apparent to those skilled in the art that other shapes of the links are suitable for use with the present invention, some suitable examples being links formed from repeating geometric shapes, such as triangles, squares, circles, polygons, arcuate shapes, parabolic shapes, oval shapes, linear shapes, and non-sinusoidal shapes. In the illustrated embodiment, the dynamic stent 100 includes a series of twenty-three (23) annular links 108 spaced equidistant from one another along the longitudinal length of the dynamic stent 100. The annular links 108 are spaced from one another such that adjacent annular links 108 are disposed in a nested relationship relative to one another, such that a crest of the sinusoidal wave of one annular link 108 is nested at least partially between a pair of troughs of the sinusoidal wave of an adjacent annular link 108.

The spacing of the annular links 108 from one another is maintained by an array of longitudinally oriented struts 110. The struts 110 are linear in form and are preferably oriented parallel with the longitudinal axis of the dynamic stent 100. The struts 110 pass through and are connected to each of the annular links 108. In the illustrated embodiment, six (6) longitudinal struts 110 are used, spaced equidistant from each other about the circumference of the dynamic strut 100. Further, although a specific number, orientation, and shape of the longitudinal struts 110 is described and depicted, it should be apparent to those skilled in the art that alternate numbers, orientations, and shapes of longitudinal struts 110 are suitable for use with and within the spirit and scope of the present invention. Although the longitudinally oriented struts 110 are depicted and described as extending continuously from one end of the dynamic stent 100 to the other, it should be apparent to those skilled in the art that the longitudinally oriented struts 110 may be intermittently disposed along the length of the dynamic stent 100, such as to connect only a few annular links 108 to one another.

As mentioned above, the dynamic stent 100 includes a permanent component 102 and a temporary component 104 which are interwoven/interlaced with one another, and in combination, to form the support frame 106. Referring to FIGS. 3 and 4, the dynamic stent 100 is depicted with the temporary component removed, leaving solely the permanent component 102. The permanent component 102 includes the longitudinal struts 110 and every other one of the annular links 108 of the support frame 106. Moreover, the permanent component 102 includes twelve (12) of the annular links 108, including the annular links 108 disposed at the ends of the dynamic stent 100.

The permanent component 102 is formed from any suitable rigid or semi-rigid material which is resistant to degradation in the body and which is compatible with the human body and bodily fluids that the dynamic stent 100 may contact. Further, preferably the dynamic stent 100 should be made from a material that allows for expansion of the dynamic stent 100 and which is able to retain its expanded shape while disposed within the lumen of the body passage. A few examples of suitable materials include stainless steel, tantalum, titanium, chromium cobalt, and nitinol.

The permanent component 102 may be formed using traditional techniques such as laser machining of tube stock, Electrical Discharge Machining (EDM), etc. The permanent component 102 may be self-expanding or balloon expandable. As should be apparent to those skilled in the art, a relatively sparse mesh pattern for the permanent component 102 may provide benefits with regard to delivery profile. A less dense mesh pattern for the permanent component 102 may mean that a ratio of a first collapsed diameter to a second expanded diameter may be smaller.

Referring to FIGS. 5 and 6, the dynamic stent 100 depicted in FIGS. 1 and 2 is shown with the permanent component removed, leaving solely the temporary component 104. The temporary component 104 includes every other one of the annular links 108 of the support frame 106. Moreover, the temporary component 104 includes eleven (11) of the annular links 108, each of the annular links 108 of the temporary component being sandwiched between a pair of adjacent annular links of the permanent component.

The temporary component 104 is formed from any suitable rigid or semi-rigid material which is amenable to degradation in the body and which is compatible with the human body and bodily fluids that the dynamic stent 100 may contact. Further, preferably the temporary component 100 is made from a material that allows for expansion of the dynamic stent 100 and which is able to retain its expanded shape while disposed within the lumen of the body passage. A few examples of suitable materials include polymers, such as the polylactide (PLA) polymer or the polymers disclosed in U.S. Pat. No. 6,461,631, the disclosure of which is hereby expressly incorporated by reference, hydrogels, and magnesium alloys. The material used may include therapeutic substances which are selectively released once the dynamic stent is implanted to aid rehabilitation of the artery wall, one such suitable material disclosed in U.S. Pat. No. 6,506,437, the disclosure of which is hereby expressly incorporated by reference.

The temporary component 104 may be formed on the permanent component 102 by dipping the permanent component 102 in a liquid polymer. The liquid polymer is then cured upon the permanent component 102. The dynamic stent 100 may then be made into custom shapes by selective physical cutting and removal of certain pieces. Other selective removal techniques may be used as well, such as laser machining. Preferably, the permanent and temporary components 102 and 104 are each formed in a geometric array, mesh, chain, interlinking pattern, etc. Preferably, the permanent and temporary components 102 and 104 are coupled to one another, such as by interconnecting and/or interweaving one to the other. Alternately, the meshwork of polymer may also be produced by lining the inside and/or outside of the permanent component 102 with a weave of polymer fibers.

In still another alternate embodiment, the temporary component 104 may be a substantially complete covering, rather than a meshwork. In still yet another embodiment, the temporary component 104 may be a substantially complete covering made of a porous, biodegradable material. The porosity may come from laser machining, from physical hole punching, or from other traditional techniques of making porous polymers.

Preferably, the temporary component 104 is formed from a biodegradable mesh of sufficient density to hold back intimal flaps and other wall/plaque components that have intruded into the lumen. The need to prop these flaps against the wall likely goes away after partial healing; presumably occurring on the order of weeks after implantation.

In light of the above description of the structure of the dynamic stent 100, the use of the dynamic stent 100 will now be described. The dynamic stent 100 is inserted within a blood vessel using well known techniques. The permanent component 102 and temporary component 104 may be delivered together into the blood vessel. Alternately, the permanent component 102 would be delivered, and then the temporary component 104 would be extruded into the artery via a catheter approach. The extrusion geometry may be a standard geometry, or a custom geometry based on the plaque geometry and composition, as imaged by intravascular ultrasound or optical coherence tomography.

As time after implant progresses, the temporary component 104 preferably degrades, resulting in a stent geometry that adjusts over time to match the changing needs of the artery wall during the remodeling process. When initially inserted, both the permanent and temporary components 102 and 104 are fully present, as shown in FIGS. 1 and 2. As time after implantation increases, the temporary component 104 degrades, resulting in the dynamic stent 100 eventually taking the form shown in FIGS. 3 and 4, wherein the temporary component 104 is absent. The temporary component 104 may be embedded with. drugs to modulate cellular reactions, a few examples being to modulate smooth muscle cell proliferation, inflammatory responses, and/or thrombus formation.

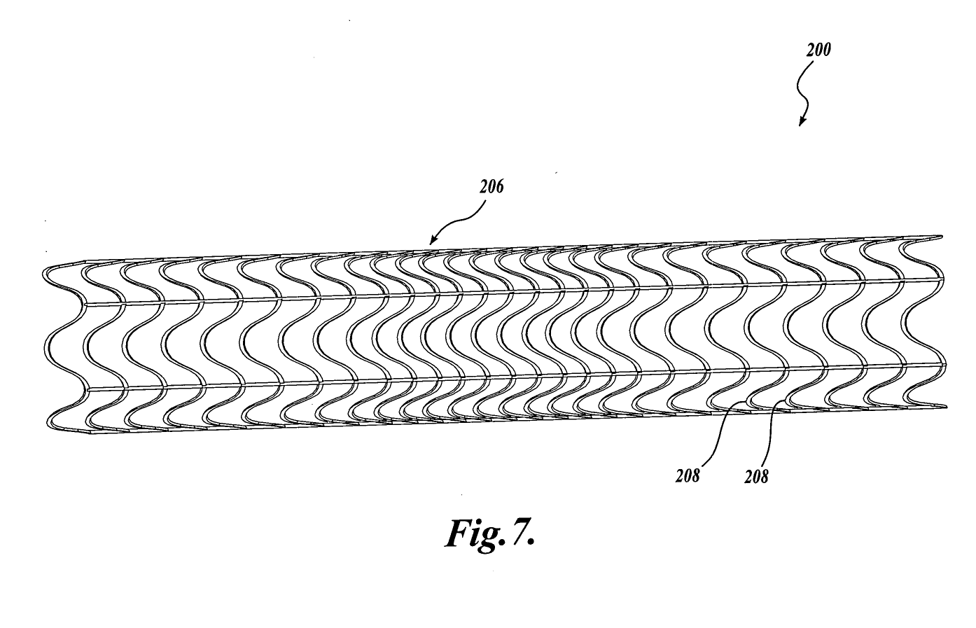

Referring to FIG. 7, an alternate embodiment of a dynamic stent 200 formed in accordance with the present invention is shown. The dynamic stent 200 is identical to the dynamic stent 100 described and depicted above with relation to FIGS. 1-6 with the exception that the stiffness of the dynamic stent 200 is variable along a length of the dynamic stent 200. In the illustrated embodiment, this accomplished by providing a support frame 206 that is non-uniform in shape. For instance, in the illustrated embodiment, the structure of the support frame 206 is modified so as to be non-uniform along its length by adjusting the spacing of the annular links 208 forming the support frame 206. More specifically, the spacing of the annular links 208 near the midpoint of the dynamic stent 200 is less than the spacing of the annular links 208 at the ends of the dynamic stent. Thus, the dynamic stent has a stiffness that is variable along the length of the dynamic stent 200, such that the stiffness at a pair of ends of the dynamic stent 200 is less than at the midpoint of the dynamic stent 200.

Referring to FIG. 2, the dynamic stent 100 depicted therein may be modified to provide variable stiffness along a length of the dynamic stent 100. This may be accomplished by forming the degradable component 104 from a plurality of degradable components, each degradable, component having a different rate of degradation. Thus, in one embodiment, the dynamic stent has a uniform stiffness along the length of the dynamic stent upon insertion into the blood vessel. However, the degradable component 104 is formed from a high rate degradable material at the ends of the dynamic stent 100 and a low rate degradable material near the midpoint of the dynamic stent 100.

After implementation, the annular links 108 of the degradable component 104 disposed at the ends of the dynamic stent 100 degrade at an elevated rate and accordingly disappear first. The annular links 108 of the degradable component 104 located at the midpoint of the dynamic stent 100 degrade at a slower rate, and therefore remain in place for a longer duration. This variable degradation of the degradable component 104 results in a variable stiffness of the support frame 106 along a longitudinal length of the support frame 106 such that a stiffness at a pair of ends of the support frame 106 is less than a stiffness at a middle of the support frame 106 after a selected period after implantation.

Although this detailed description depicts and describes two separate embodiments, wherein in one, materials of different degradation rates are used to provide variable stiffness characteristics and wherein in a second, the spacing/shape of the support frame is modified to provide variable stiffness characteristics, it should be apparent that combinations thereof are within the spirit and scope of the present invention.

While the preferred embodiment of the invention has been illustrated and described, it will be appreciated that various changes can be made therein without departing from the spirit and scope of the invention.

1. A stent comprising:
a support frame for supporting a vessel in a non-collapsed state, the support frame including;
(a) a degradable component for at least initially supporting the vessel in the non-collapsed state when the stent is first implanted in the vessel, the degradable component degradable after implantation such that support provided by the degradable component decreases a selected amount after a predetermined time after implantation; and
(b) a durable component for supporting the vessel in the non-collapsed state, the durable component resistant to degradation over time such that support provided by the durable component remains substantially constant after implantation.

2. The stent of claim 1, wherein the durable component is formed from a material selected from a group consisting of chromium cobalt, stainless steel, and nitinol.

3. The stent of claim 1, wherein the durable component is formed from a material selected from a group consisting of chromium cobalt, stainless steel, and nitinol, and wherein the degradable component is formed from a material selected from a group consisting of polymer, hydrogel, and magnesium alloy.

4. The stent of claim 1, wherein the degradable component is formed from a material selected from a group consisting of polymer, hydrogel, and magnesium alloy.

5. The stent of claim 1, wherein the degradable component or the durable component contains a therapeutic agent configured for release for absorption by a user once the stent is implanted.

6. The stent of claim 1, wherein degradation of the degradable component results in a variable stiffness of the stent along a longitudinal length of the stent.

7. The stent of claim 1, wherein the degradation of the degradable component results in a variable stiffness of the stent along a longitudinal length of the stent such that a stiffness at a pair of ends of the stent is less than a stiffness at a middle of the stent.

8. The stent of claim 1, wherein the degradable component is formed from a high rate degradable material degradable at a selected rate and a second low rate degradable material degradable at a lower selected rate.

9. The stent of claim 1, wherein the degradable component is degradable over time such that after a predetermined duration, a surface area of the support frame is decreased.

10. The stent of claim 1, wherein the support frame is formed from a plurality of struts for supporting the vessel in the non-collapsed state, some of the struts being degradable struts formed from the degradable component and some of the struts being durable struts formed from a durable material resistant to degradation.

11. The stent of claim 10, wherein at least one of the degradable struts is degradable to the extent that the degradable strut is substantially eliminated from the support frame, thereby increasing a spacing between remaining adjacent struts.

12. The stent of claim 1, wherein the support frame includes a plurality of annular links coupled to one another so as to be disposed in a nested relationship relative to one another.

13. The stent of claim 12, wherein the plurality of annular links are substantially shaped as sinusoidal waves.

14. A stent for supporting a vessel in a non-collapsed state comprising:
(a) a plurality of durable struts for supporting the vessel in the non-collapsed state; and
(b) a plurality of temporary struts for initially aiding in the support of the vessel in the non-collapsed state, wherein the temporary struts break down over time after implantation such that they no longer substantially aid in supporting the vessel in the non-collapsed state.

15. The stent of claim 14, wherein the stent includes a therapeutic agent configured for release for absorption by a user.

16. The stent of claim 14, wherein break down of the temporary struts results in a variable stiffness of the stent along a longitudinal length of the stent.

17. The stent of claim 14, wherein break down of the temporary struts results in a variable stiffness of the stent along a longitudinal length of the stent such that a stiffness at a pair of ends of the stent is less than a stiffness at a middle of the stent.

18. The stent of claim 14, wherein the temporary struts are formed from a high rate degradable material degradable at a selected rate and a second low rate degradable material degradable at a lower selected rate.

19. The stent of claim 14, wherein the durable struts and the temporary struts have outer surfaces which in aggregate form an engagement outer surface for engaging the vessel to support the vessel in the non-collapsed state, wherein the temporary struts break down over time after implantation such that the engagement outer surface decreases in surface area over time after implantation.

20. A stent for supporting a vessel in a non-collapsed state comprising:
a support frame having a durable component and a degradable component, the support frame providing a variable level of support for supporting the vessel in the non-collapsed state, wherein upon implantation, the support frame initially provides a predetermined amount of support for supporting the vessel in the non-collapsed state, and wherein the support frame changes due to exposure to environmental conditions after implantation such that after passage of a selected duration, the support frame provides a selected lessened amount of support for supporting the vessel in the non-collapsed state.

21. The stent of claim 20, wherein the degradable component at least initially supports the vessel in the non-collapsed state when the stent is first implanted, the degradable component degradable over time such that support provided by the degradable component for supporting the vessel in the non-collapsed state selectively decreases over time after implantation.

22. The stent of claim 21, wherein the durable component supports the vessel in the non-collapsed state, the durable component resistant to degradation over time such that support provided by the durable component for supporting the vessel in the non-collapsed state remains substantially constant over time.

23. The stent of claim 20, wherein the degradable component or the durable component contains medicine configured for release for absorption by a user once the stent is implanted.

24. The stent of claim 21, wherein degradation of the degradable component results in a variable stiffness of the stent along a longitudinal length of the stent.

25. The stent of claim 21, wherein the degradation of the degradable component results in a variable stiffness of the stent along a longitudinal length of the stent such that a stiffness at a pair of ends of the stent is less than a stiffness at a middle of the stent.

26. The stent of claim 21, wherein the degradable component is formed from a high rate degradable material degradable at a selected rate and a second low rate degradable material degradable at a lower selected rate.

27. The stent of claim 20, wherein the support frame is formed from a plurality of struts for supporting the vessel in the non-collapsed state, some of the struts being degradable struts formed from the degradable component and some of the struts being durable struts formed from the durable material resistant to degradation.

28. The stent of claim 27, wherein the degradable struts are degradable to the extent that they are substantially eliminated from the support frame after passage of a selected duration, thereby increasing a spacing between adjacent struts.

29. The stent of claim 20, wherein the support frame has an outer surface which engages the vessel to support the vessel in the non-collapsed state, wherein the degradable component is degradable over time such that the outer surface of the support frame decreases in surface area over time.
